(19) United States
(12) Patent Application Publication
(10) Pub. No.: US 2003/0087435 A1
(43) Pub. Date: May 8, 2003

(54) Catalytic antibodies raised against sarin; an organophosphorous anticholinesterase, and an antigen and process for the preparation thereof

(76) Inventor: Jeffrey J. Brandt, Columbus, OH (US)

Correspondence Address: Gallagher & Dawsey Co., L.P.A., Suite 226, 1487 W. Fifth Ave., Columbus, OH, 43212, US

(21) Appl. No.: 10/190,875

(22) Filed: Jul. 8, 2002

Related U.S. Application Data
(60) Provisional application No. 60/332,724, filed on Nov. 6, 2001.

(51) Int. Cl. C12N 5/06; C12N 15/08; C12N 15/06; C12N 15/07

(52) U.S. Cl. 435/451; 435/452; 435/326

(57) ABSTRACT

A system for the immune mediated in vivo hydrolysis of the nerve agent sarin, comprising metalloantibodies capable of hydrolyzing the nerve agent sarin, a cell culture method of raising these antibodies, an artificial antigen capable of stimulating the production of these antibodies, and a hapten analogue of sarin. A hapten analogue to sarin is formed by conjugating a Cr(III) trien with N^{α}, N^{ε}-di(O,O-diisopropyl) phosphoryl L-lysine (DIP). This hapten is conjugated with key limpet hemocyanin (KLH) to form an antigen capable of eliciting antibodies with reactive sites for both metals and sarin. Antibodies formed in response to this antigen are cultured in an immortal cell line and purified. The steric proximity of binding sites on the antibody for the metal trien and sarin allows hydrolyzation of the F⁻ moiety from sarin and sarin's resultant bioactive neutralization. Antibody hydrolysis activity is monitored by measurement of produced F⁻.

### RELATED APPLICATIONS

This application claims the benefit of U.S. provisional patent application Ser. No. 60/332,724, filed Nov. 6, 2001.

### TECHNICAL FIELD

The invention relates to the field of monoclonal antibodies. In particular, the present invention relates to monoclonal antibodies capable of catalytically releasing F⁻ from sarin (isopropyl methylphosphonofluoridate), an organophosphorous anticholinesterase, thereby rendering sarin inactive as a nerve agent by rendering it incapable of binding to the enzyme acetylcholinesterase.

### BACKGROUND OF THE INVENTION

Mammalian neurotransmission depends on the orderly operation of the acetycholine-acetylcholinesterase system at ganglionic, parasympathetic, and neuromuscular synaptic junctions. Acetycholine is a neurotransmitter synthesized from the precursors choline and acetyl CoA in the axonal terminal bulbs of nerves via a reaction catalyzed by choline acetyltransferase. Neurotransmission occurs when, in response to pre-synaptic stimulation, acetycholine is released from pre-synaptic vesicles and bound to post-synaptic receptors, creating an action potential, and allowing propagation of that action potential, effectively passing nerve impulses along a neural or neuromuscular pathway. After binding to the post-synaptic receptors, the neurotransmitter acetycholine is rapidly hydrolyzed by acetylcholinesterase (AChE), with principal degradation products of choline and acetic acid. The hydrolyzation process requires only 80 microseconds, making AChE one of the fastest metabolizing mammalian enzymes. Choline is a poor neurotransmitter, and the hydrolysis of acetycholine into choline effectively restores resting potential in the post-synaptic membrane, thereby terminating neurotransmission and preparing the synaptic junction for the next action potential.

Acetylcholinesterase is an enzyme occurring in nervous tissue and striated muscle and is responsible for the hydrolysis of acetylcholine (ACh). AChE has two binding sites at the active site, an anionic site and an esteric site. The anionic site is an ionized carboxyl group which interacts with the positively charged head of ACh. The esteric site is located approximately 0.7 nm from the anionic site and combines with the ester functionality of ACh. This esteric site contains three amino acids residues involved in direct binding, which are glutamic acid, serine, and alanine. The serine hydroxyl group forms a bond with the ester functionality.

Acetylcholinesterase hydrolyzes acetylcholine in two stages. In stage one, the serine hydroxyl (Ser—OH) of ACh donates electrons to the electrophilic carbon of the ACh carboxyl function. The hydrogen atom of the Ser—OH is temporarily accepted by a histidine imidazole group on the enzyme. Choline is then released leaving an acetylated enzyme. In the second stage, the transient acetylated enzyme reacts with water (H₂O) to yield acetic acid and regenerated enzyme. The imidazole ring acts as a proton acceptor to facilitate the transfer of the acetyl group to water.

Inhibitors of acetylcholinesterase are divided into two main classes according to their chemical composition and degree of stability of the formed enzyme-inhibitor (EI) complex. The first class comprises those inhibitors which have a structural resemblance to ACh and which dissociate relatively quickly from AChE. Their tendency for rapid dissociation from AChE has led to their being termed “reversible anticholinesterases.” They are used as anticholinesterase drugs to facilitate cholinergic transmission at the neuromuscular junction and at autonomic junctions with smooth muscles of the intestine, bladder, and eye. Representative compounds of this type include physostigmine, neostigmine, and pyridostigmine.

The second class, comprising those inhibitors which form long lasting stable EI complexes, are chemically unrelated to ACh and are collectively referred to as the organophosphorous anticholinesterases. While they are not truly irreversible in their chemical reaction with AChE, their very slow rate of dissociation of the phosphoryl group from AChE by hydrolysis (which can take as long as several months) has led to their being termed “irreversible” inhibitors. Representative drugs of this class include dyflos (diisopropylfluorophosphonate), parathion, malathion, and sarin (isopropyl methylphosphonofluoridate).

The organophosphorous compounds are mainly used commercially as insecticides. However, as early as the 1930's, the organophosphorous compounds were recognized as having significant potential as nerve agents for military purposes. Sarin is an organophosphate first produced in Germany in 1938. While battlefield use of sarin has been limited due to international agreements and fears of retaliation against a first-user, an increasing danger is the use of nerve agents by terrorists or rogue states. In 1995, a terrorist attack using sarin occurred in the Tokyo subway system. Eventually, 5000 persons were injured and there were 12 fatalities.

The effects of the organophosphorous anticholinesterases in the central and peripheral nervous system are a function of their ability to inhibit various esterases, particularly acetylcholinesterase. Inhibition is accomplished by phosphorylation of a serine hydroxyl group at the esteric site. Sarin and similar compounds not containing quaternary nitrogens react only with the esteric site of AChE. Initial attraction between enzyme and inhibitor is competitive and reversible. The EI complex dissociates when the concentration of inhibitor is reduced and excess ACh prevents the interaction.

The first stage of irreversible inhibition involves the removal of the X group (F⁻ in Sarin) by hydrolysis resulting in a phosphorylated enzyme at the esteric site. Hydrolysis of the phosphoryl enzyme complex by H₂O is extremely slow, but more rapid reactivation of AChE can be brought about by nucleophilic agents such as choline, hydroxylamine, or more optimally, pralidoxime, which is more potent at reactivation that hydroxylamine. The quaternary ammonium group of pralidoxime is attracted to the anionic site of AChE and holds the molecule in position so that nucleophilic attack is directed at the phosphoryl group. The antidotal effect of reactivators such as pralidoxime is most pronounced at neuromuscular junctions and less powerful at autonomic sites. It is negligible in the central nervous system (CNS) since quaternary reactivators cannot penetrate the blood brain barrier.

Acute intoxication with organophosphorous anticholinesterases results in an overload of acetycholine at neurotransmitter sites due to inactivation of the acetylcholinesterase system. The accumulation of acetylcholine at these sites results in characteristic muscarinic signs (emptying of bowels and bladder, blurred vision, profuse sweating and salivation, and stimulation of smooth muscles producing either excitatory (bronchospasm) or inhibitory (vasodilatory) reactions). At the nicotinic receptors, the effect is first excitatory and then inhibitory. The accumulation of acetylcholine at the endings of motor nerves to voluntary muscles results in powerful fasciculations of the skeletal muscle followed by paralysis. The accumulation of excessive acetylcholine in the brain and spinal cord results in characteristic central nervous system symptoms such as seizures.

Toxicity of organophosphates such as sarin is divided into acute, chronic, and long-term effects; with degree of toxicity based on the particular compound, concentration, amount absorbed, route of absorption, and types of receptor sites that are excessively stimulated. Observed effects include miosis, ocular pain, congestion of the conjuntivae, spasm of the ciliary muscle, dimness of vision, and rhinorrhea. Respiratory symptoms include dyspnea and chest tightness. The initial signs and symptoms may be followed by bronchoconstriction and excessive tracheobronchial secretions. The nicotinic effects of organophosphates are more likely to prove life threatening because of the respiratory depressant effects. Persons exposed may develop muscle fasciculations or muscle twitching followed by muscles weakness and paralysis. Death occurs due to apnea produced by respiratory muscle paralysis. In large doses, organophosphates such as sarin may produce all of the above symptoms in no well defined order. Even without apnea, in sufficient doses, sarin can cause death due to pulmonary edema and/or depression of the respiratory center, along with respiratory failure due to fasciculations or neuromuscular block of the respiratory muscles.

In lesser doses, organophosphate poisoning may present with a syndrome of headache, nausea, vomiting, abdominal cramps, and diarrhea. Moderate poisoning may present with generalized muscle weakness, skeletal muscle fasciculations, dysarthria, miosis, excessive secretions, shortness of breath, chest tightness, and dyspnea.

An “intermediate syndrome” consists of neuropathy and ocular disease with long term consequences including chronic neurologic sequelae and neurobehavioral toxicity. Low level exposure can cause a reversible down-regulation of cholinergic systems, and a range of non-cholinesterase effects that are structure specific, and do not always parallel acute toxicity. Subtle, mainly cognitive, differences between exposed and non-exposed populations are seen.

Attempts to treat acute sarin poisoning have heretofore rested substantially on a triad of prevention, supportive cardio-respiratory treatment, and pharmacologic intervention with drugs such as atropine. Prevention of sarin poisoning requires mechanical barriers, such as biohazard protection clothing and filtered breathing apparatus, which is highly expensive and cumbersome, and is unlikely to be immediately available outside of specialized military operations. Given the rapidity of respiratory and cardiovascular collapse, supportive cardio-respiratory treatment requires near immediate access to highly sophisticated medical treatment, particularly assisted ventilation, which is generally unavailable in a field situation. As a result, treatment to date has rested nearly exclusively on drugs such as atropine.

Atropine, a tertiary amine, crosses the blood brain barrier and antagonizes ACh at central muscarinic receptors, but does not inhibit the action of ACh at the nicotinic receptors. Furthermore, atropine is itself toxic in large doses. Other pharmacologic interventions include the use of oximes, a class of organic compounds with the ability to complex with acetylcholinesterase bound nerve agents, resulting in their removal from the enzyme and reactivation of normal enzymatic activity. However, the initial reaction product of sarin and AChE undergoes a process of aging, considered a second step in the continuum of irreversible inhibition, which results in the release of a leaving group from the central phosphorus atom of the nerve agent and thereby the permanent inactivation of the enzyme. This time dependent process has a half time in the sarin-acetylcholinesterase system of about five hours. After this time, oxime therapy is ineffective.

Additionally, pyridostigmine bromide, administered in advance of sarin exposure, has been shown to produce a carbamylated acetylcholinesterase in vivo that is resistant to inactivation by nerve agents. However, pyridostigmine does not afford protection without the use of other antidotes such as atropine and oximes, and is therefore considered a pretreatment adjunct rather than a pre-exposure prophylaxis or post-exposure antidote.

Therefore, what the art has needed is a means of neutralizing the danger of sarin that does not suffer the limitations of mechanical barriers to exposure, supportive therapies, or pharmacologic intervention. The present invention provides a catalytic antibody capable of rapid in vivo inactivation of sarin.

### SUMMARY OF INVENTION

A multitude of reactions can be catalyzed by enzymes composed of the naturally occurring 20 amino acids. However, these reactions would be limited were it not for the use of cofactors. These cofactors include metal ions, hemes, and flavins. Strategies that incorporate cofactors into antibody combining sites may widen the scope of antibody catalysis. One way to accomplish this goal is to design a multisubstrate analogue in which binding sites for both the cofactor and the substrate would be incorporated into the antibody with a single immunization.

This multisubstrate analogue method has been used to achieve a sequence specific cleavage of a peptide bond with Zn(II) as the cofactor. A substitutionally inert Co(III) trien was linked to a peptide substrate for immunization so that the induced binding pocket would accommodate the substrate, the trien, and the Zn(II) ion. Hydrolysis of the peptide bonds occurs through delivery of a hydroxide anion to the carbonyl carbon of the scissile amide bond of the bound substrate. Similar strategies have been applied to redox-active cofactors, such as flavins and resazurin. In addition, antibodies binding Fe(III)-mesoporphyrin IX that catalyze the H₂O₂ dependent oxidation of many different substrates have been developed and tested.

Recent studies in the area of organophosphorous anticholinesterase hydrolysis have produced metal complexes that successfully hydrolyze sarin. In particular, a Cu(II) complex of N,N,N′,N′,-tetramethylethylenediamine (tmen) and N,N,N′-trimethyl-N′-tetradecylethylenediamine (tmten) have proven to be very efficient catalysts in this regard. Catalysis arises through a complexation between the phosphoryl oxygen and the metal ion. Hydrolysis proceeds by intramolecular attack of a coordinated hydroxide ion on the bound ester, thereby releasing F⁻. Complete hydrolysis of the phosphofluoridate occurs in 700 seconds in the case of tmen, and 1200 seconds in the case of tmten. A lanthanum macrocycle catalyzes the same reaction, but on a slower basis.

In vivo, sarin binds to ACHE by removal of F⁻ from sarin and subsequently forms bonds to the esteric site of AChE. Detection of hydrolysis products in victims exposed to sarin revealed the presence of methylphosphonic acid (MPA) and isopropylmethylphosphonic acid (IMPA) in erythrocytes. Additionally, IMPA was not detected in formalin-fixed cerebellums after a period of two years, indicating that the isopropyl moiety was hydrolyzed chemically during that time.

Designing a hapten to elicit catalyzed hydrolysis requires tethering an efficient cofactor and suitable analogue of the substrate. Antibodies against sarin have been described for use in competitive inhibition enzyme immunoassays. Two artificial antigens, N^{α}, N^{ε}-di(O,O-diisopropyl) phosphoryl L-lysine (DIP)-bovine serum albumin (BSA) and DIP-KLH (key limpet hemocyanin) were synthesized. DIP is an analogue of sarin, and when linked with KLH, a protein, enables the antibodies produced to have a substrate binding pocket suitable for sarin binding. Antibodies against sarin were accordingly obtained from immunization with the DIP-KLH conjugate.

Metalloantibodies have been constructed with a coordination site for metal in the antigen binding pocket. For example, Iverson and Lerner described a method of raising antibodies to catalyze amide bond cleavage by designing a hapten as a template around which a complementary binding site would be produced with a capacity to simultaneously bind a 5-6 coordinate metal cofactor and substrate. A Co(III) trien was tethered to a small peptide in the hapten design. Monoclonal antibodies were raised possessing catalytic activity against the targeted peptide bond. It was discovered that the antibodies could accommodate a variety of trien metal complexes including Cu(II), Fe(III), and Zn(II). The Zn(II) trien was observed to be the most efficient metal cofactor complex with its highest efficiency at 1.5 mM. While it was necessary for the Zn(II) trien complex to be present for maximal activity, Zn(II), unliganded, had observable catalysis. The likeness of the trien ligand to histidine imidazoles elicited a binding pocket with Zn(II) coordinated by N's of the imidazole ligands, as histidine residues are common ligands in metalloenzymes coordinating Zn(II) at the active site. Zn(II) and other metal ions can be coordinated by cofactor binding sites in metalloantibodies through site directed mutagenesis of amino acid residues to histidines.

Accordingly, the art has needed a means for raising catalytic metalloantibodies against sarin. With these capabilities taken into consideration, the instant invention addresses many of the shortcomings of the prior art and offers significant benefits heretofore unavailable.

### BRIEF DESCRIPTION OF THE DRAWINGS

In the various figures and drawings, the following reference symbols and letters are used to identify the various elements described herein below in connection with the several figures and illustrations:

FIG. 1 shows the catalytic hydrolysis of sarin as mediated by the metalloantibodies of the present invention.

FIG. 2 shows the chemical structure of sarin (isopropyl methylphosphonofluoridate).

FIG. 3 shows the chemical structure of the DIP analogue of sarin of the present invention.

FIG. 4 shows the chemical structure of the hapten analogue of the present invention.

FIG. 5 shows the chemical structure of the antigen created by the conjugation of the hapten according to FIG. 4 with key lymphocyte hemocyanin.

### DESCRIPTION OF THE INVENTION

The detailed description set forth below in connection with the drawings is intended merely as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention.

The present invention provides monoclonal antibodies raised to catalytically release F⁻ from sarin prior to binding to AChE through the serine hydroxyl, thus preventing the binding of the sarin molecule to AChE. To do so, a hapten is synthesized which will elicit an antibody in which binding sites for the substrate and a cofactor are generated in a single immunization.

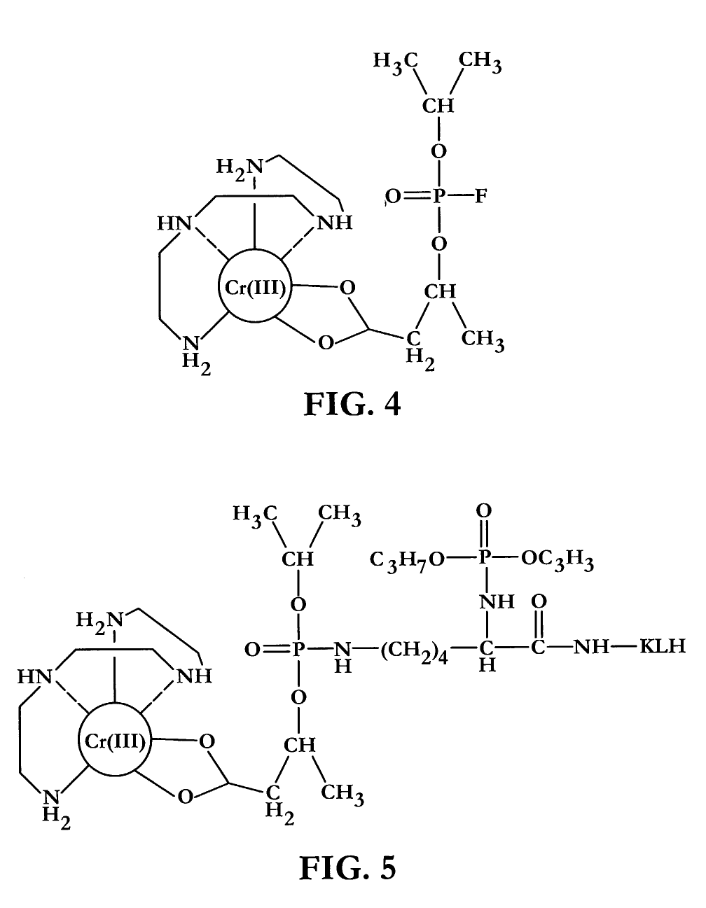

The proposed hapten, shown in FIG. 4, combines a kinetically inert Cr(III) complex with the DIP analogue, shown in FIG. 3. The Cr(III) metal has an H₂O exchange rate constant on the level of 10⁻⁶ to 10⁻² s⁻¹; therefore, it would not substitute ligands rapidly enough to be an ideal hydrolysis cofactor and would serve as a stable component of the hapten. Monoclonal antibodies are raised possessing a promiscuous binding site that would accommodate metals and/or metal complexes of kinetically labile metals such as Cr(III), Cu(II), Fe(III), Co(III) and Zn(II). The binding pockets of the catalytic antibodies brings a metal cofactor and substrate together in close proximity to facilitate a metal catalyzed hydrolysis reaction and release products.

Synthesis of the Cr(III) complex is accomplished through mixture of CrCl₃.6H2O and triethylenetetramine (trien) in ethanol/methanol. Excess solvent is then expelled through rotovaporization. Preparation of a DIP analogue is as follows: L-lysine (dissolved in .6H2O/MeOH/triethylamine) is added drop wise to diisopropyl phosphite in CCl₄, then stirred. Organic solvents are reduced and the residue extracted with ethyl acetate and acidified to substantially pH=3 with HCl. The H₂O phase is then extracted with ethyl acetate-terbutyl alcohol. The extract is then washed with NaCl solution and H₂O, and dried with MgSO₄. Solvents are evaporated and oily product is obtained. Structure determination is accomplished with phosphorous nuclear magnetic resonance (³¹P-NMR), proton nuclear magnetic resonance (¹H-NMR), and mass spectrometry (MS). A DIP-protein conjugate is obtained as follows: DIP and N-hydroxylsuccinimide are dissolved in ethyl acetate and added slowly to triethylamine containing ethyl phosphodichloridate, and stirred for approximately five hours. The reaction solution is then washed with NaHCO₃ and H₂O, then dried with MgSO₄. Solvent is removed under vacuum and oily product obtained. Structure determination is accomplished with ³¹P-NMR, ¹H-NMR and MS. Key limpet hemocyanin (KLH) is then dissolved in substantially equal parts water/DMF/triethylamine and added to the solution of the lysine ester described above in DMF, then stirred for approximately 16 hours. The solution is then dialyzed versus water for approximately three days and lyophilized. ³¹P-NMR is performed for structure determination.

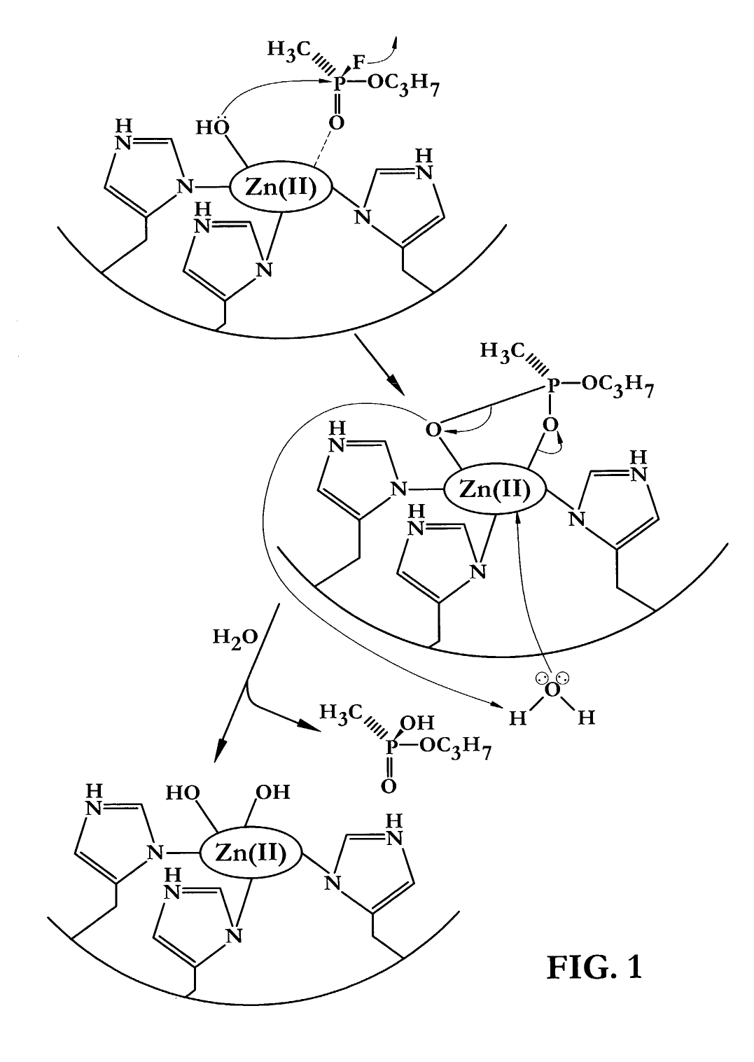

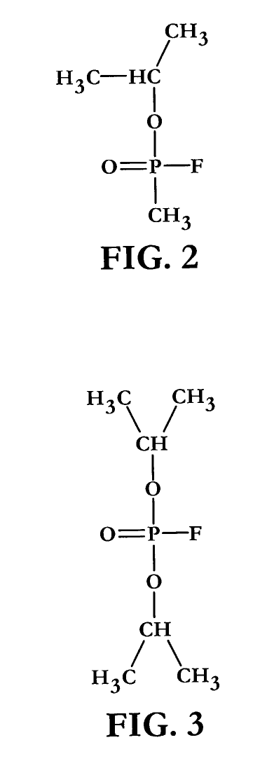

To generate antibodies, the hapten must, in general, be linked to carrier proteins typically with a spacer to avoid steric interference. To accomplish this, the Cr(III) trien is attached to the DIP-KLH analogue through a carboxylate function as shown in FIG. 5. Methyl-3-hydroxybutyrate is then added to the phosphoryl group via condensation. The ester functionality is demethylated and the Cr(III) complex is added over heat and stirring. Excess solvent is removed by slow evaporation at substantially 25° C. The hydrolysis reaction elicited by the catalytic antibodies, as it takes place in vivo, is shown in FIG. 1.

Mice are immunized with the antigen. The mouse spleen cells serve as a source of antibody producing normal parent cells. In order to produce homogenous antibodies (Abs) against the antigen, cultured mouse myeloma cells are fused with the spleen cells using a surface-active agent such as, by way of example and not limitation, inactivated Sendai virus. The resulting hybrid clones grow in vitro as a cultured hybridoma line. Hybridoma cell lines secreting antibodies are cultured and the colonies are screened using an enzyme-linked immunosorbent assay (ELISA) for their ability to bind selectively to the original antigen and not the carrier protein. Antibody purification is performed by affinity chromatography on a protein G column.

Efficiency of the antibody in hydrolyzing sarin is monitored and measured by fluorescence titration studies performed to determine an upper limit dissociation constant (K_{d}). The K_{d} will indicate the binding affinity of the antibody for substrate. The antibody binding pockets contain tryptophan residues whose florescence is quenched upon active site titration with products and hapten, therefore a decrease in fluorescence indicates hapten binding.

Kinetic experiments are performed on sarin to ascertain the Kₘ (Michaelis-Menton constant) and k_{cat} (catalytic rate constant) parameters. A fluoride ion electrode is used to monitor the increase in F⁻ ion concentration versus time. As the reaction progresses, the F⁻ increases and eventually levels off in an asymptotic manner. A ratio of k_{cat}/k_{uncat} indicates the efficiency of the antibody as a catalyst for the hydrolysis of substrate.

These variations, modifications, alternatives, and alterations of the various preferred embodiments, arrangements, and configurations may be used alone or in combination with one another as will become more readily apparent to those with skill in the art with reference to the following detailed description of the preferred embodiments and the accompanying figures and drawings.

Numerous alterations, modifications, and variations of the preferred embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the present invention are described herein, it is to be understood that the practice of such additional modifications and variations and the equivalents thereof, are within the spirit and scope of the invention as defined in the following claims.

The corresponding structures, materials, acts, and equivalents of all means or step plus function elements in the claims below are intended to include any structure, material, or acts for performing the functions in combination with other claimed elements as specifically claimed.

I claim:

1. A method of making cells useful for the production of monoclonal antibodies, the method comprising fusing an antibody secreting B-lymphocyte with an immortal cell to form a fusion product thereof.

2. A method according to claim 1, wherein the immortal cell is a myeloma cell.

3. A method according to claim 1, wherein the immortal cell is a mouse myeloma cell.

4. A method according to claim 1, wherein the B lymphocyte is a spleen cell.

5. A method according to claim 1, wherein the B lymphocyte is a mouse spleen cell.

6. A cell for producing a plurality of antibodies, comprising a fusion product of a B-lymphocyte and an immortal cell.

7. A cell for producing a plurality of antibodies according to claim 6, wherein the B-lymphocyte is a spleen cell.

8. A cell for producing a plurality of antibodies according to claim 6, wherein the B-lymphocyte is a mouse spleen cell.

9. A cell for producing a plurality of antibodies according to claim 6, wherein the immortal cell is a myeloma cell.

10. A cell for producing a plurality of antibodies according to claim 6, wherein the immortal cell is a mouse myeloma cell.

11. A cell for producing a plurality of antibodies according to claim 6, wherein the antibodies produced by the antibody producing cell bind to sarin esteric site.

12. A cell for producing an plurality of antibodies according to claim 6, wherein the antibodies produced are a plurality of metalloantibodies with a metallic element selected from the group consisting of Chromium, Iron, Zinc, Copper, and Cobalt.

13. A cell culture consisting essentially of a plurality of antibody producing cells comprising a fusion product of a B-lymphocyte and an immortal cell.

14. A monoclonal antibody capable of binding to a sarin esteric site.

15. A monoclonal antibody according to claim 14, wherein the antibody is a metalloantibody with a metallic element selected from the group consisting of Chromium, Iron, Zinc, Copper, and Cobalt.

16. A method of producing a plurality of monoclonal antibodies, comprising the culturing of at least one antibody producing cell comprising a fusion product of a B-lymphocyte and an immortal cell in a medium under conditions suitable for the production of the plurality of monoclonal antibodies therein, and collecting the plurality of monoclonal antibodies from the medium.

17. A composition comprised of a plurality of antibodies, wherein essentially all of the plurality of antibodies bind to a common epitope.

18. A composition according to claim 17, wherein the common epitope is a sarin esteric site.

19. A composition according to claim 17, wherein the plurality of antibodies is selected from the group consisting of IgG and IgM antibodies.

20. A method for producing an antigenic hapten resembling a chemical structure of sarin, comprising:
synthesis of a Cr(III) complex;
synthesis of a DIP conjugate; and
conjugating the Cr(III) complex and the DIP conjugate.

21. An antigenic hapten according to claim 20.

22. A method for producing an antigen analogue of sarin, comprising:
attachment of an antigenic hapten (Cr(III)-DIP conjugate) to a key lymphocyte hemocyanin molecule.

23. The antigen analogue of sarin according to claim 22.

24. A method for producing a plurality of antibodies to sarin, comprising:
immunization of a mouse with the antigen analogue according to claim 23;
fusion of at least one spleen cell from the immunized mouse with at least one cultured mouse myeloma cell to produce a plurality of fused cells producing a plurality of antibodies;
culturing the fused cells in vitro;
collection of the plurality of antibodies produced by the fused cells; and
purification by affinity chromatography of the plurality of antibodies thereby produced.
